(19) United States
(12) Patent Application Publication
(10) Pub. No.: US 2014/0155774 A1
(43) Pub. Date: Jun. 5, 2014

(54) NON-INVASIVELY DETERMINING RESPIRATION RATE USING PRESSURE SENSORS

(71) Applicant: The Regents of the University of California, Oakland, CA (US)

(72) Inventors: Majid Sarrafzadeh, Anaheim Hills, CA (US); Lauren Samy, Los Angeles, CA (US); Amir Vajid, Redondo Beach, CA (US)

(21) Appl. No.: 14/094,476

(22) Filed: Dec. 2, 2013

Related U.S. Application Data
(60) Provisional application No. 61/732,219, filed on Nov. 30, 2012.

(51) Int. Cl. A61B 5/00; A61B 5/08

(52) U.S. Cl. CPC A61B 5/7278; A61B 5/0816; A61B 5/7267; A61B 5/7207; A61B 5/746; A61B 5/7282; A61B 5/725; A61B 5/6892; A61B 5/0004
USPC 600/529

(57) ABSTRACT

A respiration rate of a human subject is derived by: (1) receiving data corresponding to a sequence of two-dimensional pressure distributions of the human subject as applied against an array of pressure sensors over time; (2) converting the data into a one-dimensional waveform; (3) identifying oscillations in the one-dimensional waveform as occurrences of breath of the human subject; and (4) deriving the respiration rate of the human subject based on frequencies of the oscillations.

### CROSS-REFERENCE TO RELATED APPLICATION

This application claims the benefit of U.S. Provisional Application Ser. No. 61/732,219 filed on Nov. 30, 2012, the disclosure of which is incorporated herein by reference in its entirety.

### FIELD OF THE INVENTION

This disclosure generally relates to determining respiration rate and, more particularly, determining respiration rate using pressure sensors.

### BACKGROUND

The monitoring of respiration rate is of importance for various clinical applications such as the detection and monitoring of sleep disorders, monitoring newborns for Sudden Infant Death Syndrome (“SIDS”), as well as identifying patients at high risk up to 24 hours before adverse events like strokes and cardiac arrests. As important as respiration rate is, its measurement is often inaccurate or even neglected altogether. In a typical hospital setting, respiration rates are typically measured by direct observation by medical personnel or by attaching sensors to patients' bodies if continuous monitoring is desired. In the former case, the measurement is often inaccurate due to the different ways of measuring respiration rate. Measuring the respiration rate of a patient using a stethoscope, for example, can yield different results than manually tracking the movement of a patient's chest. Discrepancies are also reported between counting breaths for 30 seconds and multiplying by 2 and counting breaths for 60 seconds. Also, if patients are aware that their respiration rate is being monitored, they may control their breathing, resulting in an inaccurate measurement. In the latter case, a patient might suffer from skin irritation or discomfort.

It is against this background that a need arose to develop the non-invasive system and method to objectively and accurately measure respiration rate described herein.

### SUMMARY

Embodiments of this disclosure provide a non-invasive system and method for monitoring respiration rate. In some embodiments, the system is used while a patient is sleeping. While the patient is lying down on top of a bed mattress or a bed sheet embedded with pressure sensors, the system derives and displays the respiration rate of the patient. This can be used to identify irregular breathing, such as that associated with sleep disordered breathing. The system can also detect non-respiratory movement and episodes of apnea. When apnea is detected, the system can alert a medical professional or a care provider. Pressure-related information is logged and can be reviewed later by a medical professional. A user interface developed as part of some embodiments provides multiple features, such as a replay feature that facilitates the task of reviewing a patient's logged respiratory activity. The system can be used in both home and clinical environments. At home, embodiments can be used to remotely monitor a patient's breathing patterns. Also, the system can be used in a clinical environment, such as a sleep study center.

In some embodiments, pressure sensors incorporated in a bed mattress or a bed sheet are fabric-based pressure sensors. Their small size and low cost render these pressure sensors desirable for inclusion in large numbers in a dense sensor array. Because these sensors are embedded in a fabric, they are also comfortable for patients. Pressure data collected from the sensors can be transmitted to a processing unit for analysis. In some embodiments, pressure data is transmitted to a local, client computer and from there to a server computer to be filtered and further processed. The results of data analysis can be displayed to the patient as well as made available to care providers. The care provider can be alerted if the respiration rate falls out of an acceptable range, indicating an apneic episode, the onset of a cardiac arrest or heart attack, or other events for which respiration rate can provide an early warning.

In some embodiments, raw analog pressure data is digitized and transmitted to a processing unit for analysis. In the processing unit, the digitized form of the raw pressure data can be dynamically filtered for different users and applications. The pressure data can be processed by classification of oscillations as breaths or non-respiratory movement. In some embodiments, a machine learning procedure can be used to provide accurate measurements in real time. To differentiate noise from actual breaths, the machine learning procedure can be used in real time to capture a particular patient's breathing patterns so that a system can adapt to his or her breathing pattern specifically. In such manner, the system can be trained to adapt to an individual's sleeping patterns to provide more accurate respiratory information over time. Unlike analog signal processing techniques, a machine learning procedure can improve the outcome of the system as more training data becomes available from pressure sensors.

In some embodiments, data collected from an array of pressure sensors is digitized and transmitted over a serial bus to a processing unit. Signals that represent respiratory activity are extracted from the digitized data. When a patient inhales and exhales during respiratory activity, diaphragm movements are made, which translate to changes in pressure applied to the sensor array or a subregion of the sensor array. Thus, to capture these respiratory movements, the sum of all, or a subset of, pressure sensor signals from the sensor array is derived and used as a signal to represent respiratory activity.

In some embodiments, low-pass filtering is performed on signals to remove any high-frequency noise that is collected from pressure sensors. Additionally, a filter to compensate for a logarithmic drift of the pressure sensors is included so that pressure data can be adjusted according to a “zero” level (e.g., a ground level) between inhale and exhale pressure exertions.

Using identified breaths, a respiration rate can be derived and displayed by dividing the number of breaths that occurred over a time interval in which they occurred. Once breaths are identified and a respiration rate is derived, a procedure for determining apnea can be implemented. When a certain threshold of time has passed without a breath or if the respiration rate falls below a threshold, a system can determine that an episode of apnea is being experienced by a patient. This information can be logged, and an alarm can be triggered to notify a care provider.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1 shows an architecture of a respiration rate monitoring system, according to an embodiment of this disclosure.

FIG. 2 shows a textile sensor array, according to an embodiment of this disclosure.

FIG. 3 shows a circuit for scanning a textile sensor array, which can be included in a data sampling unit in accordance with an embodiment of this disclosure.

FIG. 4 shows a processing unit implemented in accordance with an embodiment of this disclosure.

FIG. 5 shows a screenshot generated by a user interface module in accordance with an embodiment of this disclosure.

FIG. 6 is a Table setting forth multiple states of a respiration rate monitoring system, according to an embodiment of this disclosure.

FIG. 7(a) shows a plot of unfiltered sum of pressure data, and FIG. 7(b) shows a plot of localized sum of pressure data, according to an embodiment of this disclosure.

FIG. 8 shows a plot of pressure data with a best-fit line that models a logarithmic drift of pressure sensors, according to an embodiment of this disclosure.

FIG. 9 shows a plot of sum of frames over time with annotations added as vertical lines at corresponding frame numbers that breaths were observed, according to an embodiment of this disclosure.

FIG. 10 shows a general flow of a respiration rate detection procedure and associated data pre-processing and filtering, according to an embodiment of this disclosure.

FIG. 11 shows a plot including a high frequency noise oscillation that precedes a breath, according to an embodiment of this disclosure.

FIG. 12 shows a plot including a small amplitude oscillation that corresponds to an actual breath, according to an embodiment of this disclosure.

FIG. 13 shows a state machine of system state, according to an embodiment of this disclosure.

### DETAILED DESCRIPTION

Respiration rate is an important indicator of a person's overall health. It is important for many clinical uses and is often monitored during clinical evaluations as one of the four standard vital signs along with temperature, heart rate, and blood pressure. Respiration rate is important for detecting sleep disordered breathing like sleep apnea or SIDS, and is also a significant predictor of deterioration, cardiac arrest, and urgency for admission to an intensive care unit (“ICU”). An abnormally high respiration rate, such as greater than 24 breaths/min, is observed in patients who experienced cardiac arrests or have been admitted to the ICU. A high respiration rate also is observed to correlate with a high mortality rate. In unstable patients, relative changes in respiration rate can be greater than changes in heart rate or blood pressure, thus making respiration rate a better and more accurate indicator for discriminating between stable patients and patients at risk.

Because of its importance for many clinical uses, being able to non-invasively, accurately, and continuously measure respiration rate is of significant value. Embodiments of this disclosure provide a system that uses a fabric-based (or textile) pressure sensor array for detecting a patient's respiration rate. The system can be used in a home setting, a clinical setting, or both. The pressure sensor array can be incorporated in, for example, a bed mattress or a bed sheet, and the system can continuously detect respiratory rate, movement, and the presence of a patient on the mattress or bed sheet. While the patient is sleeping freely and comfortably without sensors attached to his or her body, the system can non-invasively collect, analyze, and display respiration-related data. Respiration rate can be extracted via analyzing time-stamped pressure map or distribution sequences. A bed mattress or a bed sheet is a desirable human-sensor interface for measuring respiration rate because of its non-invasive nature and also because the long amount of time that patients and people in general spend lying in bed, which allows for a lengthy monitoring period. Sleep is also an important time period for monitoring respiration rate since many emergencies associated with a high respiration rate, such as cardiac arrest and heart attacks, are likely to occur during the last phase of sleep.

### System Architecture

FIG. 1 shows an architecture of a respiration rate monitoring system, according to an embodiment of this disclosure. The system can be implemented for respiration rate monitoring in a clinical setting of a hospital or for daily use at a home, where a patient can be remotely monitored.

Referring to FIG. 1, the system includes three main components: a textile sensor array 100, a data sampling unit 102, and a processing unit 104. The textile sensor array 100 captures a two-dimensional pressure distribution or map of a patient as the patient lies on or otherwise applies force or pressure on the texture sensor array 100, and the data sampling unit 102 collects sensor outputs corresponding to the pressure distribution and conveys the sensor outputs to the processing unit 104 via wireless or wired transmission. The processing unit 104 analyzes the sensor outputs for respiration rate monitoring and other applications. Specifically, the processing unit 104 executes or otherwise performs procedures described in the following sections.

Although the textile sensor array 100, the data sampling unit 102, and the processing unit 104 are shown as separate components in FIG. 1, it is contemplated that these components can be combined or further sub-divided in other embodiments. It is also contemplated that processing performed by the processing unit 104 also can be performed in whole or in part by another component, such as the data sampling unit 102 or a remote server computer.

According to a specific implementation of the architecture of FIG. 1, a bed mattress incorporates a 2.5 m×1.25 m textile sensor array of 64×32 pressure sensors. This dense array of pressure sensors can provide true positive rates and false positive rates that are vast improvements from results of other approaches using a smaller number of sensors. A data sampling unit is connected to the textile sensor array and performs matrix scanning to collect pressure map sequences. Retrieved pressure map signals from the pressure sensors are quantified to a digitized form, whose values range from 0 to 255, with 0 representing no pressure and 255 representing maximum pressure. The data sampling unit interfaces with a computer's serial port, and data is transferred over an Universal Asynchronous Receiver/Transmitter (“UART”). Another serial bus, such as an Universal Serial Bus (“USB”) also can be used. For every “0xA” data packet transmitted from the computer, a 64×32 frame of pressure sensor data is received. The data transfer from the data sampling unit to the computer is determined to be between about 550 ms and about 600 ms. To mitigate against tight timing and to allow the receipt of clean data, a period of about 666 ms was chosen, which allows a refresh rate of about 1.5 Hz. This low sampling rate is determined to be sufficient for the application at hand. According to the Nyquist rate, the sampling rate to avoid aliasing is equal to twice the highest frequency present in a signal, or f_{N}=2B. For a refresh rate of about 1.5 Hz, the highest frequency that can be correctly represented is about 0.75 Hz. This allows the detection of a maximum breathing rate of about 45 breaths/minute. This upper bound on respiratory rate is sufficient for the application at hand, since even newborns, with their small lung capacity and resulting frequent breathing, have a respiratory rate that typically does not exceed 40 breaths/min. A higher or lower sampling rate can be used for other implementations.

Textile Sensor Arrays:

Textile sensor arrays are desirable for monitoring applications because of their flexibility, light weight, and cost-effectiveness. A textile sensor array can be implemented using a textile sensor sheet that exhibits a piezoresistive effect, namely an electrical resistance of the sensor sheet varies in response to an applied force or pressure. In some embodiments, a textile sensor sheet can be implemented using textile fibers (e.g., synthetic or natural fibers) that are individually coated with an electrically conductive material, such as an electrically conductive polymer or a polymer with electrically conductive additives dispersed therein, and then knitted, woven, interlaced, bonded, or otherwise combined to form the sensor sheet. Examples of suitable electrically conductive polymers include nitrogen-containing aromatic polymers (e.g., polypyrroles, polycarbazoles, polyindoles, polyanilines, and polyazepines), sulfur-containing aromatic polymers (e.g., poly(3,4-ethylenedioxythiophene)), polythiophenes, polyfluorenes, polyphenylenes, polypyrenes, polyazulenes, polynapthalenes, polyacetylenes, and poly(p-phenylene vinylene). In other embodiments, a textile sensor sheet can be implemented using a pre-formed textile sheet, such as a woven or non-woven textile sheet, which is then coated, impregnated, or otherwise combined with an electrically conductive material to form the sensor sheet. During use, an initial resistance between a top surface and a bottom surface of a textile sensor sheet can be high, as a natural structure of the sensor sheet can be a relatively loose collection of fibers that are spaced by air gaps. When force or pressure is applied to either, or both, of the surfaces of the sensor sheet, interior fibers can be pressed together, thereby lowering the resistance. Other implementations of a textile sensor sheet are contemplated, such as by leveraging a piezoelectric effect in place of, or in conjunction with, a piezoresistive effect.

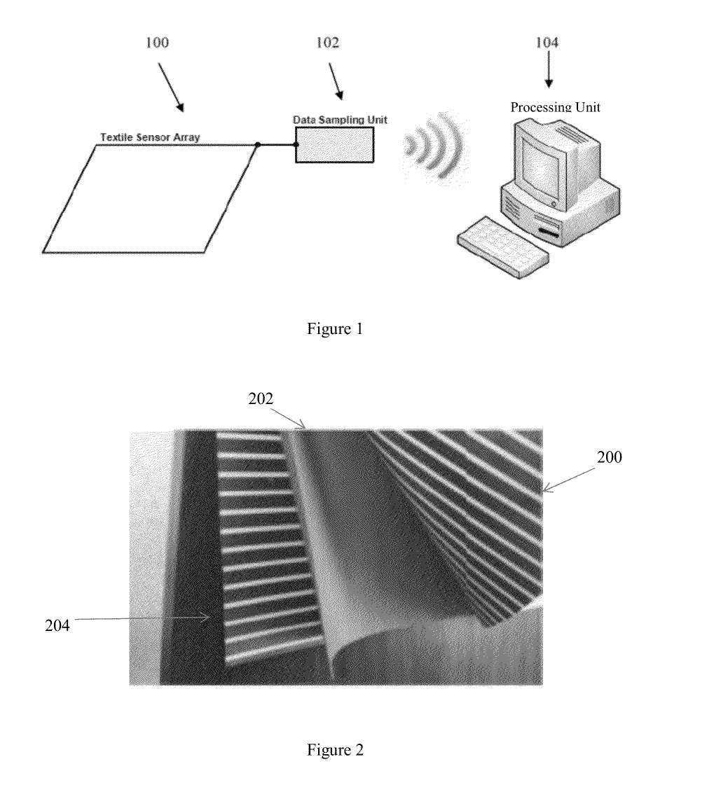

FIG. 2 shows a textile sensor array, according to an embodiment of this disclosure. The sensor array is implemented as a three layer, stacked structure, in which a middle layer 202 is a textile sensor sheet that is disposed between and laminated, bonded, or otherwise combined with a top layer 200 and a bottom layer 204. In the illustrated embodiment, the top layer 200 and the bottom layer 204 can be implemented using woven or non-woven textile sheets that are coated with an electrically conductive material to form elongated conductive strips. Specifically, these elongated strips correspond to conductive buses that are substantially parallel to one another in the top layer 200 and are substantially parallel to one another in the bottom layer 204. Suitable electrically conductive materials include electrically conductive polymers, polymers with electrically conductive additives dispersed therein, metals, and metal alloys, and suitable coating techniques include deposition, spraying, printing, and roll-to-roll coating. As shown in FIG. 2, the conductive buses of the top layer 200 are oriented relative to the conductive buses of the bottom layer 204 so as to cross over one another at crossing points or intersections. In the illustrated embodiment, the conductive buses of the top layer 200 are substantially orthogonal to the conductive buses of the bottom layer 204, although other crossing angles are contemplated, such as from about 1° to about 90°, from about 5° to about 90°, from about 20° to about 90°, from about 45° to about 90°, from about 90° to about 179°, from about 90° to about 175°, from about 90° to about 160°, or from about 90° to about 135°. Each intersection of a top conductive bus and a bottom conductive bus sandwiches a portion of the textile sensor sheet, thereby forming a pressure sensor at that location. In such manner, an array of pressure sensors is formed as an M×N matrix of pressure sensors, where M is a total number of the conductive buses of the top layer 200, N is a total number of the conductive buses of the bottom layer 204, and M×N is the total number of pressure sensors. In general, M can be the same as or different from N, and each of M and N can be 1 or more. To improve accuracy of respiration rate detection, the sensor array desirably includes at least 25 pressure sensors, at least 50 pressure sensors, at least 100 pressure sensors, at least 500 pressure sensors, at least 1000 pressure sensors, at least 2000 pressure sensors, at least 3000 pressure sensors, at least 4000 pressure sensors, at least 5000 pressure sensors, at least 6000 pressure sensors, at least 7000 pressure sensors, or at least 8000 pressure sensors, and up to 10000 pressure sensors or more.

While textile sensor arrays allow the seamless integration of electronic elements in fabric to create flexible embedded systems, they also can pose challenges. For example, a signal-to-noise ratio (“SNR”) can be relatively low in a textile sensor array. This can make it challenging to extract meaningful features from data without proper filtering of the data. Another challenge can be cross-talk occurring between pressure sensors. When pressure is applied to one sensor, this pressure also can be sensed by other sensors around it, resulting in a false response and inaccurate pressure values. Also, sensor drift can be an inherent property of pressure sensors that can cause a signal to grow logarithmically over time until a system reaches operating temperature. Unless accounted for, such drift can impede accuracy of respiration rate detection. In order to compensate for these challenges that are often characteristic of textile sensor arrays, efficient procedures are implemented to mitigate against their influence and provide high accuracy for respiration rate monitoring purposes.

Data Sampling Unit:

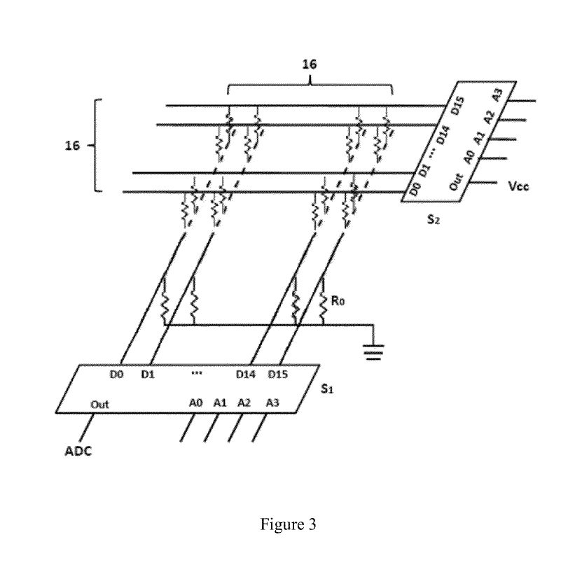

FIG. 3 shows a circuit for scanning a textile sensor array, which can be included in a data sampling unit in accordance with an embodiment of this disclosure. Each conductive bus on a bottom layer of the sensor array is connected to an analog-to-digital converter (“ADC”) via an analog switch module S1 and to ground via an offset resistor Ro. Each conductive bus on a top layer of the sensor array is connected to a voltage supply V_{cc} via an analog switch module S2. The analog switch modules S1 and S2 operate in conjunction to selectively address a particular pressure sensor, and a scanning sequence is synchronized by a microcontroller (not shown), which also can be included in the data sampling unit. For example, when S2 connects a selected bus i on the top layer to V_{cc} and S1 connects a selected bus j to the ADC, the ADC can read a voltage through a pressure sensor located at an intersection of bus i and bus j, namely located at row i and column j, which voltage can be denoted as Vᵢⱼ. In such manner, the circuit has random accessibility for reading an arbitrary sensor within the sensor array. Also, the single ADC can be shared among multiple sensors, thereby reducing manufacturing burden and facilitating large-scale sensing applications relative to alternative implementations in which each sensor has a separate ADC to sample a sensor output.

Processing Unit:

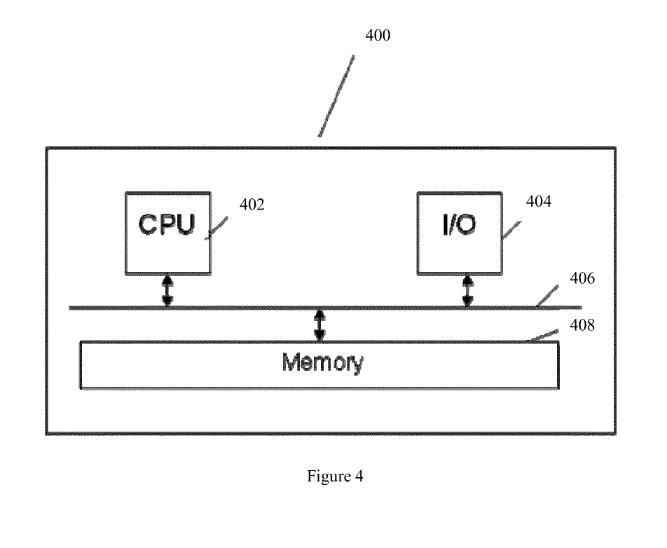

FIG. 4 shows a processing unit 400 implemented in accordance with an embodiment of this disclosure. Depending on a specific application, the processing unit 400 can be implemented as, for example, a portable electronics device, a client computer, or a server computer. Referring to FIG. 4, the processing unit 400 includes a central processing unit (“CPU”) 402 that is connected to a bus 406. Input/Output (“I/O”) devices 404 are also connected to the bus 406, and can include a keyboard, mouse, display, and the like. An executable program, which includes a set of software modules for the procedures described in the following sections, is stored in a memory 408, which is also connected to the bus 406. The memory 408 can also store a user interface module to generate alerts or other visual presentations of respiration rate.

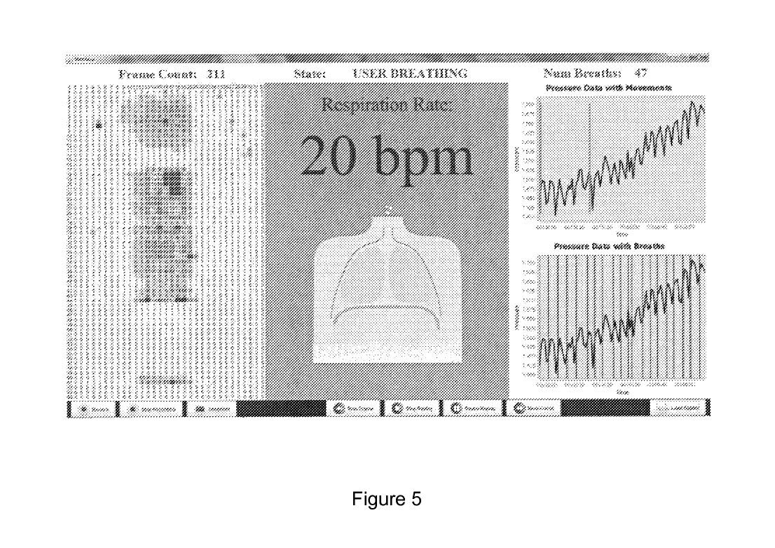

A screenshot generated by a specific implementation of a user interface module is shown in FIG. 5, according to an embodiment of this disclosure. The left panel displays an array of pressure sensors in a 32×64 grid that matches a layout of a textile sensor array, which is incorporated in a bed mattress. The value shown in each cell is a 2-character hexadecimal representation of a data byte collected from a corresponding sensor in the sensor array. The cells are color-coded to make it easy to distinguish pressure values of significantly different magnitudes. More specifically, the difference between two sequential color thresholds can be selected to be a factor of 2. This allows for a clear visualization of a pressure distribution of a patient on the bed mattress as shown in FIG. 5.

The panel on the right side includes two plots that graph pressure data over time and display events detected by a respiratory rate monitoring system substantially in real time. The data in each plot is a moving window of the last one minute. The bottom plot includes vertical lines or other indications for each breath detected by the system. The top plot includes a vertical line or other indication when the system detects that the patient has moved, and another line when the system identifies that the patient is stable again. The procedures for determining when these lines are displayed are described in the following sections.

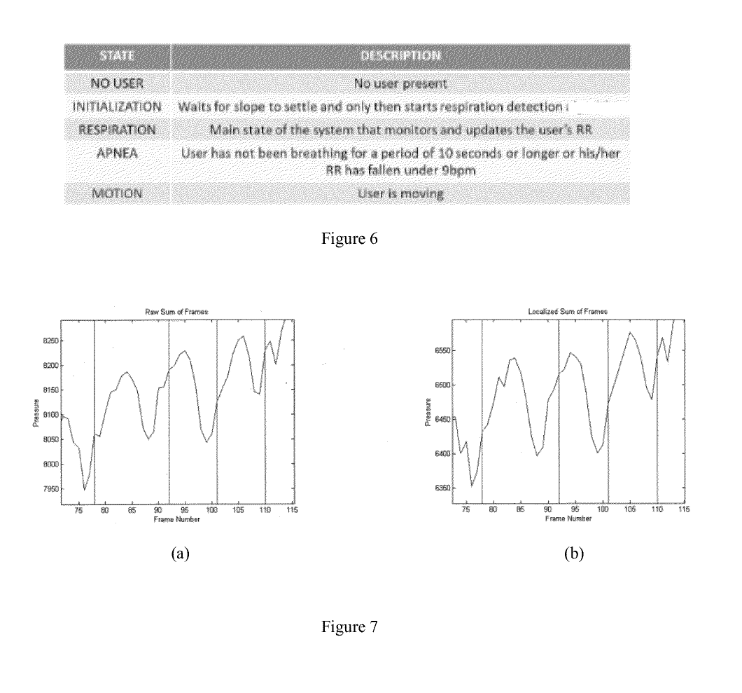

The top portion of the screenshot shows a current frame number, a state of the system, and number of breaths detected. The frame number updates every time a new pressure data frame is collected from the bed mattress. Similarly, the number of breaths updates whenever the system identifies that a breath has occurred. The system state is displayed as one of multiple states set forth in the Table shown in FIG. 6. The procedures that determine a current state of the system are also described in the following sections.

The center panel displays a current respiration rate of the patient as derived by the system. It is updated at about 1.5 Hz frequency, which matches a sampling rate of the system. There is also an animation of the patient breathing in the center panel, which is triggered when a new breath is identified. This helps to visualize the patient's breathing pattern.

Lastly, the bottom panel includes buttons that assist in recording and replaying logs. Recorded log files can be replayed by the system for review by the patient or a medical professional. The system can replay logged data back at the same frequency at which it was collected.

### Data Formatting and Recording

In some embodiments, a respiratory rate monitoring system records pressure data collected from a textile sensor array over time into a log file. When this record function is initiated, a comma-separated-value (“CSV”) file with a time-stamped name is generated. From this point on, any new frame (e.g., 32×64 grid of pressure data) read from the sensor array is output to the file. More specifically, for each frame, the value of each individual pressure sensor is recorded as an integer and in the following format. Each new row of a frame starts at the beginning of a new line, and individual values of a single row are separated by commas. An additional blank line is printed between each data frame. The result is a CSV file that can be imported and analyzed as a three-dimensional array (frame number, row, and column). Other formats can be used in place of, or in combination with, a CSV file format.

To test the operation of the system in identifying features that represent breaths, some type of ground truth can be synchronized with the data recording format of the system. The ground truth mechanism can identify every breath a patient makes so that breaths can be extracted from collected data during post-analysis review. This can be achieved through manual annotation by an observer as well as through a video recording of a sleeping patient.

An annotation can be made whenever a patient breathes, since this can be observed by tracking the movement of the patient's chest. To simplify the annotation and allow synchronization with the recorded data, a module is designed so that the observer can press a button on a computer every time a breath is observed. The system then logs these events by saving frame numbers of the annotated breaths to an additional log file. This allows correlation of observed breaths to pressure data frames being logged.

To synchronize video recordings with the logged data, a current system frame number can be included with each recorded video frame. This way, a video recordings can be reviewed during post-analysis to observe any abnormal data and synchronize it to the actual frame number it occurred. Video recordings can be used to identify any unexpected changes in pressure (e.g., during movements) as well as any possible breaths that the annotator might have missed.

### Data Analysis

This section explains how collected data is analyzed and processed to detect different system states as well as the procedures used to transition between those states. In some embodiments, prone position of patients can be analyzed for respiration rate, although supine and side positions also be analyzed in other embodiments.

Feature Selection:

A task to be performed in guiding analysis involves determining which feature (or set of features) should be extracted from pressure data collected from a textile sensor array. A particular feature selected is a sum of an entire frame. This feature is selected since movements made on a bed mattress or a bed sheet, including that of a diaphragm during respiration, should affect the overall sum of the frame. As further explained in the sections that follow, this frame-sum signal is useful for identifying different kinds of movements, from actual bodily movements to respiratory activity to a lack of respiratory activity.

A localized sum can be considered as another input feature for respiration characterization. A comparison can be made whether a localized sum that focuses on the chest, upper body, or entire body of a patient can provide cleaner data. FIG. 7(a) shows an example of a frame-sum signal that is chosen for analysis, while FIG. 7(b) shows a localized sum over time. The localized sum shown is determined by specifying a rectangular box that outlines upper and middle regions of a body and calculating the sum of pressure values inside of this box. As seen in FIG. 7(b), the localized sum has additional noise near a (local) maximum at frame 84. There is an additional minimum at frame 82, which does not appear in the unfiltered sum, as shown in FIG. 7(a). This may result from cross-talk in a sensor array. More specifically, some pressure data that is part of respiratory activity can be affecting sensors outside of a patient's outline and vice versa. In the examples shown, the entire sum of frame is less affected by cross-talk, and, thus, can be a desirable feature to use for respiration rate monitoring.

NO USER—Initial System State:

The NO USER state is an initial state of a system which indicates that no patient is sensed by a textile sensor array. The system remains in the NO USER state as long as a sum of pressure values in a current frame is below a pre-determined threshold. From P=F/A, where F is a weight of a patient and A is a surface area covered by his or her body, the threshold for the NO USER state can be derived from pressure values of a patient with the lightest expected weight (e.g., 45 kg). To account for a larger range of weight variation, the threshold can be determined to be a fraction (e.g., one third) of the threshold of the lightest expected weight. In other embodiments, the threshold can be individually adjusted according to a weight of a particular patient.

Initialization:

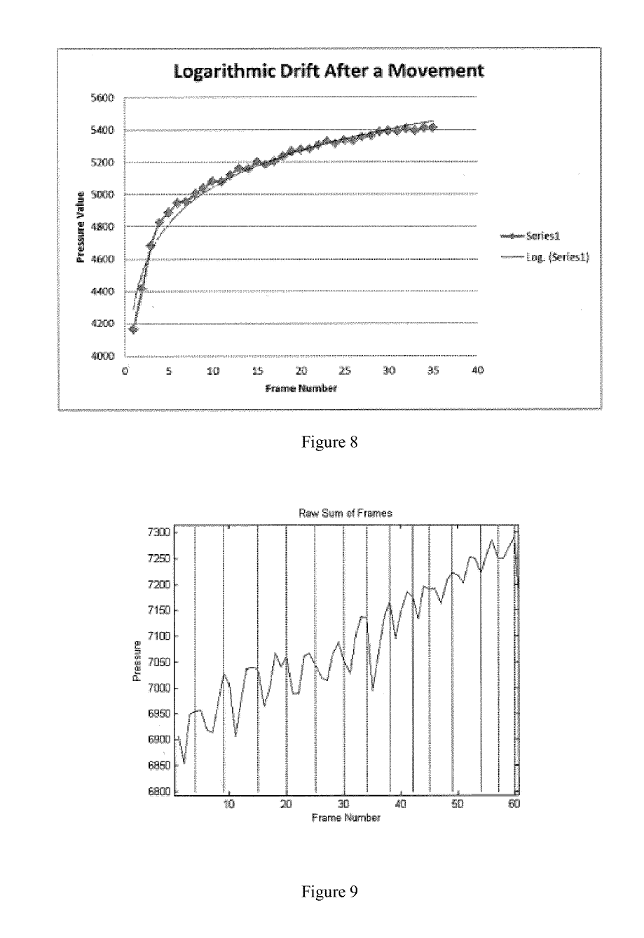

Once a patient lies on the textile sensor array and thereby exceeds the pressure threshold, the system state changes to the INITIALIZATION state, during which the system initializes itself to prepare for detecting breaths. During initialization, the system waits for a slope of an incoming logarithmic signal to stabilize or reach a substantially constant value. This logarithmic behavior can result from drift in pressure sensors as a consequence of their implementation technology. FIG. 8 shows an example of an occurrence of logarithmic drift for a patient's pressure data. In some embodiments, a first-order derivative procedure for digital signal processing can compensate for the drifting and project voltage signals onto a horizontal plane.

After the slope has stabilized, the system starts the breath detection procedure where it actively monitors for breaths. After successfully detecting 3 breaths within a time interval of 20 seconds (or another time interval), corresponding to a minimum breathing rate of 9 breaths/min, the system enters the RESPIRATION state. This threshold ensures that human beings, rather than heavy objects, can trigger the transition to the RESPIRATION state.

RESPIRATION—Detecting Patients' Breaths:

The RESPIRATION state is entered once a stable representation of a patient's breaths is identified. It can be entered from either the INITIALIZATION state or the MOTION state. A stable signal of a relatively small average slope (e.g., after drifting compensation) is desired in order to identify a low-SNR respiratory activity signal. The frequency of a frame-sum signal can be selected as a feature that represents a respiration rate of the patient. FIG. 9 shows a section of an example frame-sum signal with additional vertical lines to show synchronized breath annotations made by an observer. As can be seen, each period of the signal has an annotation marking that a breath occurred in that time interval. Thus, breaths occur at substantially the same frequency as the frequency of the frame-sum signal. This means that a single oscillation of the frame-sum signal represents a single breath made by the patient. This can be understood since a breath exerted by the patient involves an inhalation period followed by a period of exhalation, which correspond to increases and decreases in pressure exerted onto the sensor array. Therefore, accurate identification of these oscillations can be used to identify breaths.

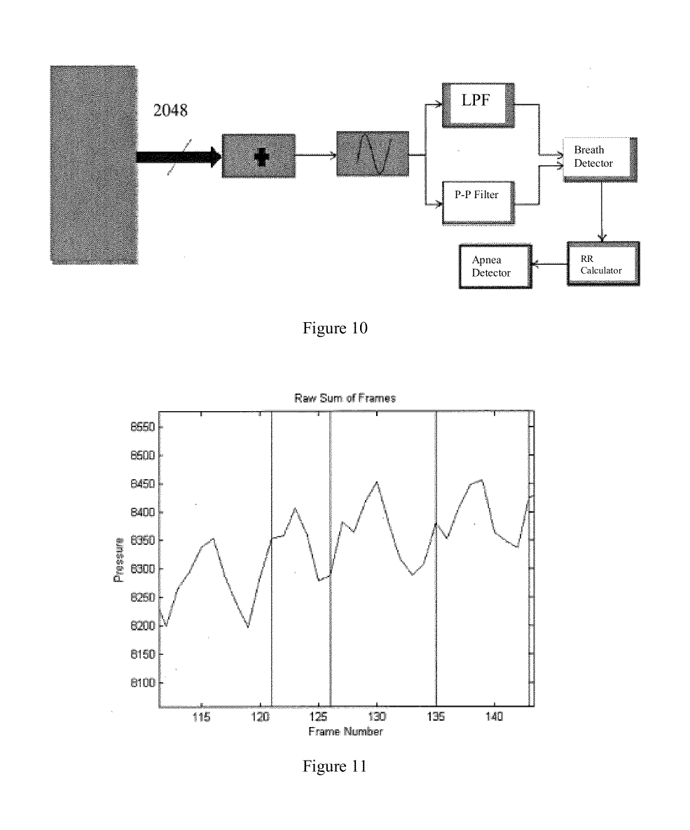

A procedure is developed to capture the occurrences of these oscillations, and to derive an instantaneous respiration rate and determine if a patient is suffering from apnea. A flow chart of the procedure and associated data pre-processing and filtering is shown in FIG. 10. Starting from the left side of the flow chart, raw pressure data is summed by a summing module and fed to a waveform module as time series data, such as a one-dimensional time series data over time or frame numbers. The waveform module identifies a waveform through peak detection. The result is then fed to a low-pass filter and a peak-to-peak amplitude filter. The results of these filters are then fed to a breath detection decision module, which can derive and update a respiration rate, which can be used to detect apnea. Although not shown in FIG. 10, another filter can be included to compensate for a logarithmic drift of pressure sensors. The explanation of the individual modules of the flow chart is set forth in the following.

The waveform module is used to identify respiratory oscillations. To do this, a peak detection procedure is developed to identify both maxima and minima of a waveform. The frequency of this waveform as well as its peak-to-peak amplitude are derived to create filters to distinguish noise from actual breaths. The oscillation period is measured by counting the number of frames from a maximum to its corresponding minimum that followed. The peak-to-peak amplitude is measured as a pressure value difference from a maximum to its corresponding minimum.

In a specific implementation of the low-pass filter, a cutoff frequency of the filter is set to 3 frames per oscillation (30 breaths/min) so that any 2-frame oscillations can be an indicator of possible noise. An example of this type of noise in the system is shown in FIG. 11. As can be seen, small oscillations next to actual breath oscillations should be ignored as noise. Another cutoff frequency can be selected for other embodiments, such as one that is adjusted to account for higher typical respiration rates of newborns and infants. However, the frequency of oscillations may not provide enough information alone for filtering, since two oscillations found directly next to each other as in FIG. 11 (one noise and one breath) should be classified properly. The system should be able to ignore the noise while still properly classifying the breath, even though together they create a high frequency oscillation. Thus, a peak-to-peak amplitude filter is also introduced to the system as a second classifier in distinguishing between noise and true respiratory activity.

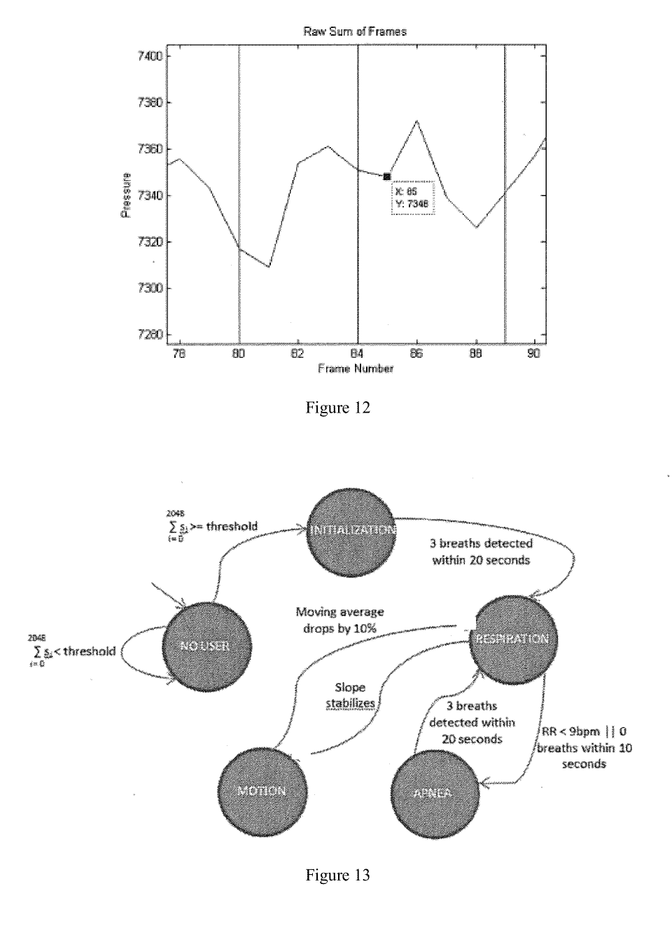

The peak-to-peak amplitude of each oscillation is recorded so that small amplitude oscillations can be ignored as noise while large amplitudes can be classified as breaths. In a specific implementation, a small amplitude can be considered to be within a particular fraction of a patient's typical amplitude, such as within 50%, within 40%, within 30%, within 20%, within 10%, or less. However, the peak-to-peak amplitude of oscillations alone may not provide enough information to identify an occurrence of noise. A breath potentially can have a relatively small peak-to-peak amplitude that would be ignored as noise with the peak-to-peak amplitude threshold filter alone. Specifically, when a small peak-to-peak amplitude oscillation is observed as a breath, a horizontal distance (corresponding to a time interval) from maximum to minimum can be greater than or equal to 2 frame times. Occurrences of this situation coincide with low frequency oscillations, while occurrences of noise coincide with a high frequency oscillation. An example of a small peak-to-peak amplitude breath is shown in FIG. 12. As shown in FIG. 12, the breath oscillation has a small peak-to-peak amplitude because the actual minimum or maximum of the oscillation was not sampled, namely it was between frame samples. Thus, the actual peak-to-peak amplitude would have been greater than a selected peak-to-peak amplitude threshold. Thus, occurrences of small amplitude, low frequency oscillations should be classified by the breath detection decision module as breaths, while high frequency oscillations with similar amplitudes should be considered noise.

In some embodiments, a machine learning procedure can be used to classify oscillations as breaths or otherwise detect respiratory activity. To differentiate noise from actual breaths, the machine learning procedure can be used in real time to capture a particular patient's breathing patterns so that the system can adapt to his or her breathing pattern specifically. In such manner, the system can be trained to adapt to an individual's sleeping patterns to provide more accurate respiratory information over time. Contemplated approaches for breath detection include template-based procedures, a Naive Bayes Network to train data and select features for classification, eigenvectors, Bayesian networks, logistic regression, nearest neighbor procedures for classification, as well as other types of machine learning procedures.

Once the breath detection decision module has detected breaths, the next operation is performed by a respiration rate calculator module to derive a current respiration rate of a patient. The current respiration rate can be calculated as a moving average over a particular time interval, such as over the last 30 seconds (45 frames). A data structure can be created to include occurrences of breaths over the last 45 frames, which can be used to display the respiration rate to an end user. It is desirable to derive the rate over a recent time interval so that any deviations in the rate can be quickly identified (e.g., for apnea). In a specific implementation, when the respiration rate falls below a selected threshold of 9 breaths/min, the system transfers state from RESPIRATION to APNEA.

APNEA—Respiratory Cessation:

To enter the APNEA state, a few options are possible. One way to enter this state occurs if a current respiration rate of a patient falls below 9 breaths/min or another selected threshold. If the patient's healthy respiration rate is naturally high, however, it can take some time for the respiration rate (which is measured, for example, over the last 30 seconds) to reach 8 breaths/min or lower. Therefore, a second way to enter this state occurs if no breaths are detected over a particular time interval, such as 10 seconds. This time interval can be selected because sleep apnea is clinically defined as the complete cessation of airflow for at least 10 seconds.

To reenter the RESPIRATION state, the system detects at least a particular number of breaths over a particular time interval, such as at least 3 breaths over 20 seconds. This threshold is selected to correspond to a breathing rate of at least 9 breaths/min.

MOTION—Detecting Patients' Movements:

The system includes functionality for movement detection, such as in the breath detection decision module. Movements can be classified as such by a machine learning procedure, or can be identified by a drop in a sum of pressure sensor data. Movements are important to be identified for at least two reasons. First, non-respiratory movements should be compensated or filtered so that they are not confused with respiratory activity. Second, the system should re-calibrate itself after a patient has moved. This is because modified pressure signals can experience a restarted logarithmic drift and because the patient's breath signal can be modified in the new position. Thus, the system adapts to the patient's movement to accurately extract the patient's breaths.

In order to detect patients' non-respiratory movements (which can interfere with accurate detection of respiratory activity), a specific implementation of the system uses a moving average of a sum of pressure values over a particular time interval, such as over the last 5 frames. This is used to represent an average pressure a patient exerts on the sensor array for his or her current position. Unless the patient is moving, this average follows the logarithmic drift increase that is expected from fabric-based pressure sensors. Thus, any deviation from the logarithmic drift increase can be identified.

Significant non-respiratory movements, which can interfere with determination of respiration rate in the RESPIRATION state, can involve a deviation of at least a particular fraction from the current calculated moving average, such as at least 10% drop from the current calculated moving average. Thus, this deviation can be selected as a threshold for classifying a decrease as a non-respiratory movement.

After a movement occurs, the system remains in the MOTION state until the sensor array stabilizes. Similar to the INITIALIZATION state, the system waits until the sensor array's logarithmic drift levels off to an expected slope. However, in this case, the system can recover more quickly than during the INITIALIZATION state because typically a subset of sensors, namely the ones that are affected by the movement, should be updated.

Also, different from the INITIALIZATION state, breaths do not have to be detected to move the system out of the MOTION state. This is because no assumption is made that the patient is actually breathing when he or she is making a movement while sleeping. Thus, the system waits until the slope of the logarithmic drift reaches a stable value and transitions to the RESPIRATION state to start detecting breaths again.

In other embodiments, movements can be classified as such by a machine learning procedure. The tuning performed by the machine learning procedure can aid in identifying and filtering out non-respiratory movement.

A state machine summarizing the transitions between the states explained above is shown in FIG. 13.

An embodiment of this disclosure relates to a non-transitory computer-readable storage medium having computer code thereon for performing various computer-implemented operations. The term “computer-readable storage medium” is used herein to include any medium that is capable of storing or encoding a sequence of instructions or computer codes for performing the operations, methodologies, and procedures described herein. The media and computer code may be those specially designed and constructed for the purposes of this disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as floptical disks; and hardware devices that are specially configured to store and execute program code, such as application-specific integrated circuits (“ASICs”), programmable logic devices (“PLDs”), and ROM and RAM devices. Examples of computer code include machine code, such as produced by a compiler, and files containing higher-level code that are executed by a computer using an interpreter or a compiler. For example, an embodiment of the disclosure may be implemented using Java, C++, or other object-oriented programming language and development tools. Additional examples of computer code include encrypted code and compressed code. Moreover, an embodiment of this disclosure may be downloaded as a computer program product, which may be transferred from a remote computer (e.g., a server computer) to a requesting computer (e.g., a client computer or a different server computer) via a transmission channel. Another embodiment of this disclosure may be implemented in hardwired circuitry in place of, or in combination with, machine-executable software instructions.

While the invention has been described with reference to the specific embodiments thereof, it should be understood by those skilled in the art that various changes may be made and equivalents may be substituted without departing from the true spirit and scope of the invention as defined by the appended claims. In addition, many modifications may be made to adapt a particular situation, material, composition of matter, method, operation or operations, to the objective, spirit and scope of the invention. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the invention. Accordingly, unless specifically indicated herein, the order and grouping of the operations is not a limitation of the invention.

What is claimed is:

1. A non-transitory computer-readable storage medium, comprising executable instructions to:
receive data corresponding to a sequence of two-dimensional pressure distributions of a human subject as applied against an array of pressure sensors over time;
convert the data into a one-dimensional waveform;
identify oscillations in the one-dimensional waveform as occurrences of breath of the human subject; and
derive a respiration rate of the human subject based on frequencies of the oscillations.

2. The non-transitory computer-readable storage medium of claim 1, wherein the executable instructions to convert the data into the one-dimensional waveform include executable instructions to:
for each two-dimensional pressure distribution of the sequence of two-dimensional pressure distributions, sum pressures values collected from at least a subset of pressure sensors included in the array of pressure sensors.

3. The non-transitory computer-readable storage medium of claim 1, further comprising executable instructions to:
apply a low-pass filter to the one-dimensional waveform.

4. The non-transitory computer-readable storage medium of claim 1, further comprising executable instructions to:
apply a peak-to-peak amplitude filter to the one-dimensional waveform.

5. The non-transitory computer-readable storage medium of claim 1, further comprising executable instructions to:
process the one-dimensional waveform to compensate for logarithmic drift in pressure values collected from the array of pressure sensors.

6. The non-transitory computer-readable storage medium of claim 1, wherein the executable instructions to identify the oscillations in the one-dimensional waveform include executable instructions to:
classify each oscillation as an occurrence of breath based on at least one of a frequency of the oscillation and an amplitude of the oscillation.

7. The non-transitory computer-readable storage medium of claim 1, wherein the executable instructions to identify the oscillations in the one-dimensional waveform include executable instructions to:
classify each oscillation as an occurrence of breath based on both a frequency of the oscillation and an amplitude of the oscillation.

8. The non-transitory computer-readable storage medium of claim 1, wherein the executable instructions to identify the oscillations in the one-dimensional waveform include executable instructions to:
apply a machine learning procedure to the one-dimensional waveform to identify the oscillations as the occurrences of breath.

9. The non-transitory computer-readable storage medium of claim 1, wherein the executable instructions to identify the oscillations in the one-dimensional waveform include executable instructions to:
identify an occurrence of non-respiratory movement of the human subject; and
identify the oscillations in the one-dimensional waveform by omitting a portion of the one-dimensional waveform corresponding to the occurrence of non-respiratory movement.

10. The non-transitory computer-readable storage medium of claim 1, wherein the executable instructions to derive the respiration rate include executable instructions to:
derive the respiration rate as corresponding to a moving average of the frequencies of the oscillations over a specified time interval.

11. The non-transitory computer-readable storage medium of claim 1, further comprising executable instructions to:
generate an alert if the respiration rate falls outside of a specified range.

12. A non-transitory computer-readable storage medium, comprising executable instructions to:
receive time series data corresponding to a sequence of pressure maps of a human subject as applied against a sensor sheet over time;
identify an occurrence of non-respiratory movement of the human subject;
omit a portion of the time series data corresponding to the occurrence of non-respiratory movement;
identify an oscillation in a remaining portion of the time series data as an occurrence of breath of the human subject; and
derive a respiration rate of the human subject based on a frequency of the oscillation.

13. The non-transitory computer-readable storage medium of claim 12, further comprising executable instructions to:
apply a low-pass filter to the time series data.

14. The non-transitory computer-readable storage medium of claim 12, further comprising executable instructions to:
apply a peak-to-peak amplitude filter to the time series data.

15. The non-transitory computer-readable storage medium of claim 12, further comprising executable instructions to:
process the time series data to compensate for logarithmic drift in pressure values collected from the sensor sheet.

16. The non-transitory computer-readable storage medium of claim 12, wherein the executable instructions to identify the oscillation include executable instructions to:
classify the oscillation as the occurrence of breath based on at least one of the frequency of the oscillation and an amplitude of the oscillation.

17. The non-transitory computer-readable storage medium of claim 12, wherein the executable instructions to identify the oscillation include executable instructions to:
apply a machine learning procedure to the time series data to identify the oscillation as the occurrence of breath.
